(19) United States
(12) Patent Application Publication
(10) Pub. No.: US 2016/0129097 A1
(43) Pub. Date: May 12, 2016

(54) METHOD OF PROCESSING A VETERINARY TUMOR VACCINE AND A VETERINARY TUMOR VACCINE PROCESSING KIT

(71) Applicants: JEREMY DELK, Nicholasville, KY (US); DONALD COHEN, Nicholasville, KY (US); JOHN YANNELLI, Nicholasville, KY (US)

(72) Inventors: JEREMY DELK, Nicholasville, KY (US); DONALD COHEN, Nicholasville, KY (US); JOHN YANNELLI, Nicholasville, KY (US)

(21) Appl. No.: 14/934,690

(22) Filed: Nov. 6, 2015

Related U.S. Application Data
(60) Provisional application No. 62/075,946, filed on Nov. 6, 2014.

(51) Int. Cl. A61K 39/00

(52) U.S. Cl. CPC A61K 39/0011; A61K 2039/552; A61K 2039/55561; A61K 2039/572; A61K 2039/55516; A61K 2039/5152

(57) ABSTRACT

The present invention provides methods for preparing and administering a tumor vaccine. The processes include resecting tumors and isolating tumor cells, followed by treatment with ultraviolet radiation to activate the tumor cells. Cells can then be mixed with an unmethylated single stranded oligodeoxynucleotide chain adjuvant to formulate a vaccine and then administered to a subject. The vaccines can be autologous, allogeneic or hybrids thereof to the subject.

### RELATED APPLICATIONS

This application claims priority to U.S. Provisional Patent Application 62/075,946, filed Nov. 6, 2014, which is hereby incorporated by reference in its entirety.

### TECHNICAL FIELD

The present invention relates generally to the field of veterinary medicine, and more particularly to a method and kit for processing a veterinary tumor vaccine.

### BACKGROUND

In 2012, there were approximately 70 million dogs and 74 million cats in the United States. On average, it is estimated that approximately 6 million (each) of these animals will be diagnosed with cancer each year and up to 25% of the positively diagnosed dogs will die. Cancer is widespread among aging animals. For example, 50% of all dogs over the age of 10 will develop cancer, and 32% of all cats will die from cancer. Accordingly, there is a need in the art for improved treatment methods and devices for addressing the problem of cancerous tumors in cats and dogs and in other veterinary applications. Needs for advances in the art include more effective cancer vaccines, synthetic adjuvants targeted to veterinary species, and therapeutic strategies which work in conjunction with existing treatments including surgery, radiation therapy and chemotherapy.

### SUMMARY OF THE INVENTION

In accordance with the purposes described herein, a method is provided for processing an autologous/allogeneic/hybrid veterinary tumor vaccine. The method may be broadly described as including the steps of: (a) resecting a tumor, (b) dissociating tumor cells, (c) activating tumor cells, (d) mixing the activated tumor cells with an adjuvant (a material designed to enhance the anti-tumor immune response) to form a tumor vaccine, and (e) injecting the tumor vaccine into an animal. The tumor cells may be mixed with a defined tumor cell line derived from the animal prior to activation. The tumor cells may be mixed with cells derived from a different or secondary tumor derived from the subject.

In a second embodiment, the method further includes placing the tumor in a sterile container with sterile medium plus a panel of antibiotics and anti-mycotics to clean the tumor tissue.

In another embodiment, the dissociating of the tumor cells further includes dissecting the tumor to remove necrotic tissue and residual normal tissue, placing the tumor in a digestion medium, and stirring the mixture until a single cell suspension is obtained. In a preferred embodiment, the digestion medium is a mixture of collagenase, hyaluronidase and DNAase.

In a further embodiment, activation of a tumor vaccine includes treating tumor cells with ultraviolet radiation, which “activates” a form of tumor cell death, making them more immunogenic for processing by immune cells.

In other embodiments, the method may also include the steps of cryopreserving the tumor vaccine and/or shipping the tumor cell vaccine to a veterinarian.

In accordance with the purposes described herein, a kit for processing veterinary tumor vaccines is provided.

The present invention also provides for an adjuvant comprising 20-50 unmethylated deoxynucleotides linked by phosphorothioate bonds. The sequence of the adjuvant may comprise at least one CpG motif. The sequence may comprise between one and four AACGTT motifs. The sequence may comprise a palindromic stem-loop of at least two AACGTT motifs.

The present invention further provides for culturing isolated tumor cells in vitro. Isolated tumor cells may be combined with defined cell lines or with cells isolated from other tumors prior to activation.

The present invention also provides, in part, for combination therapy comprising administering the vaccine along with monoclonal antibodies to PD-L1, PD-1 and/or CTLA-4.

### BRIEF DESCRIPTION OF THE DRAWINGS

FIG. 1 shows an example of one possible veterinary species-optimized CpG ODN. The example shown is one of several possible designs for CpG ODN adjuvants that are optimized for stimulation of immune cells in veterinary species. The example shown is optimized for canine and displays the oligodeoxynucleotide sequence and the predicted secondary structure of the molecule. Sequence motifs that are important to targeting adjuvant activity to veterinary species (canine in this example) are highlighted in the FIGURE.

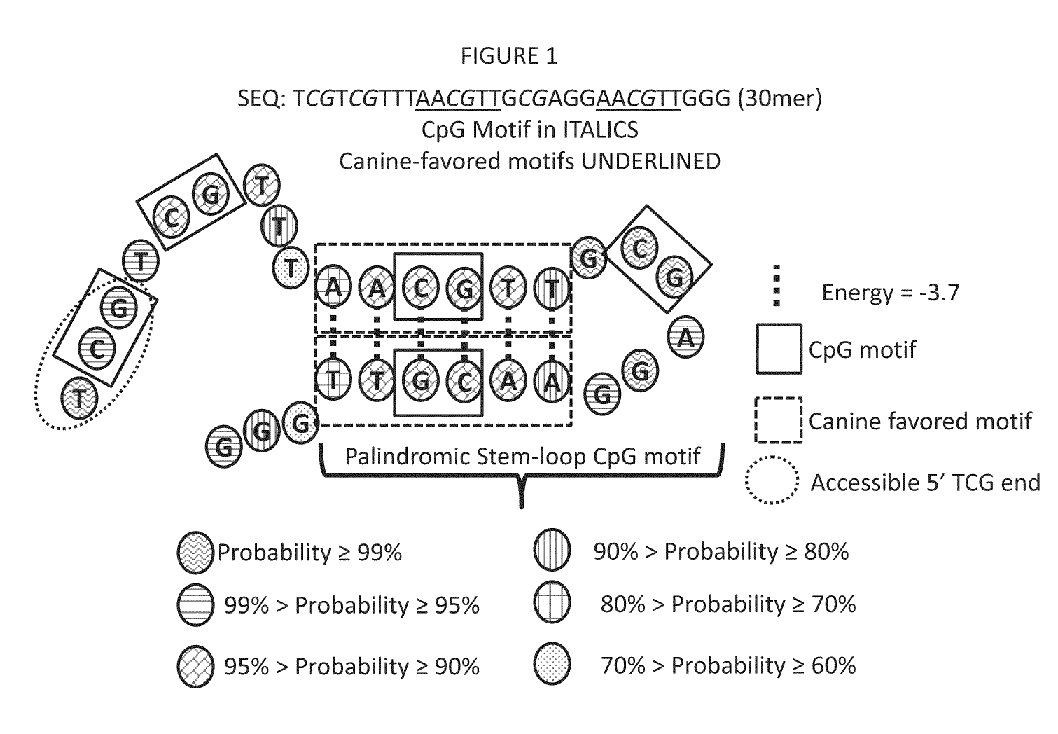

### DETAILED DESCRIPTION

The present invention provides in part for methods of generating an immune response to tumor cells within a subject comprising administering activated tumor cells to a subject. The tumor cells may be autologous, allogeneic or a hybrid thereof to the subject. The method comprises obtaining tumor cells from a subject or from a defined cell line, optionally adding further tumor cells, such as those derived from a different tumor or from a defined cell line, activating the tumor cells, adding an adjuvant to the activated tumor cells to formulate a vaccine and administering the vaccine to the same subject. The method may alternatively comprise administering the vaccine to a different subject. The method may further comprise the steps of: (a) resecting a tumor from a subject, (b) dissociating tumor cells, (c) activating a tumor vaccine, (d) mixing the tumor vaccine with an adjuvant (a material designed to enhance the anti-tumor immune response), and (e) injecting the tumor vaccine into an animal. The animal may be the same as the subject.

The tumor cells may be derived or obtained from any tumor or combinations thereof. For example, cells may be derived from cutaneous tumors (i.e. melanoma, sarcomas and mast cell) and visceral tumors (i.e. colon, lung, prostate, kidney) or other tumors of the following general histologic types: carcinoma, sarcoma, myeloma, lymphoma, leukemia. The tumor cells may be a combination of tumor cells derived from different or multiple tumors within the subject or comprise isolated tumor cells mixed with defined tumor cell lines, such as tumor cells lines from the same species and/or same tumor type as that of the subject.

The methods of the present invention may further comprise placing a resected tumor or cells obtained therefrom in a sterile container with sterile medium plus a panel of antibiotics and anti-mycotics to clean the tumor tissue.

The methods of the present invention provide in part for obtaining tumor cells. Dissociating of the tumor cells may comprise dissecting a tumor to remove necrotic tissue and residual normal tissue, placing the tumor in a digestion medium, and stirring the mixture until a single cell suspension is obtained. In some embodiments, the digestion medium may comprise a mixture of collagenase (Type IV), hyaluronidase and DNAase (Type I). Concentrations of enzymes in the digestion medium may comprise collagenase IV (about 1 mg/ml), hyaluronidase (about 0.1 mg/ml) and DNAse 1 (about 10 ug/ml).

The methods of the present invention further comprise the step of activation of isolated tumor cells. Activation may comprise treating tumor cells with ultraviolet radiation (about 25,000 microjoules of UV-A radiation), which “activates” or initiates or triggers signaling within the tumor cells to undergo a form of programmed tumor cell death (apoptosis). The process of activation provides for changes within the tumor cells that render the cells more immunogenic for processing by immune cells when administered to a subject.

In certain embodiments, the method may also comprise the steps of cryopreserving the tumor vaccine and/or shipping the tumor cell vaccine to a veterinarian. The cells may be cryopreserved either prior to or post activation.

### Adjuvant

The present invention provides in part for an adjuvant and methods for preparing an adjuvant for mixing with activated tumor cells to formulate a tumor vaccine. The adjuvant may comprise a synthesized single stranded oligodeoxynucleotide chain (ODN) in which all nucleotides are unmethylated and all nucleotides are linked together by phosphorothioate bonds. The length of the oligodeoxynucleotide chain comprises between about 20 to 50 deoxynucleotides. The length of the ODN may comprise about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 deoxynucleotides.

The ODN of the adjuvant may further comprise several dinucleotides internally within the ODN sequence as a motif (“CpG”) consisting of an unmethylated cytosine triphosphate deoxynucleotide (“C”) in the 5′ position followed by an unmethylated guanosine triphosphate deoxynucleotide (“G”) in the 3′ position. The di-deoxynucleotide consisting of a C followed by a G is generally referred to as “CpG motif”. The 5′ end of the ODN sequence begins with an unmethylated thymidine triphosphate deoxynucleotide (T) followed by a CpG motif. This tri-deoxynucleotide (TCG) has been shown to be important for the immunostimulatory activity of this class of CpG ODN adjuvant (Putta, et al. Bioconjugate Chem. 2010, 21, 39-45).

In further embodiments, the adjuvants ODN may comprise additional internal CpG regions that comprise the sequence AACGTT (recited from 5′ to 3′) which features two adenine deoxynucleotides “AA” at the 5′ end, followed by a CpG motif and then two thymidine deoxynucleotides “TT” at the 3′ end. The sequence AACGTT has been identified as important for stimulation of immune cells (e.g., canines as set forth by Ren et al., Vaccine 28 (2010) 2458-2464). The number of AACGTT sequences within the ODN is at least one, but may be as many as four.

In some embodiments, the ODN sequence comprises at least one palindromic stem-loop sequence or motif having two AACGTT segments. The stem-loop sequence may also have at least one CpG motif in the loop region of the sequence in addition to being present within the AACGTT motifs. The stem-loop structure has been identified as adding to stability and improved immunostimulation for immune cells from several species, including canines. (Yang, et al. International Immunopharmacology 15 (2013) 89-96).

In further embodiments, the adjuvant may be optimized for adjuvant activity in certain animal species, including but not limited to canine and feline. Those skilled in the art will appreciate that optimization may be achieved by varying certain aspects of the adjuvant, such as length, number of CpG motifs, the location of a stem-loop, as well as the size of the loop within the stem-loop. Some versions of the veterinary species-optimized adjuvant may be chemically modified such that the CpG ODN becomes amphipathic in nature which allows any of the following:
Enhanced uptake of the CpG ODN adjuvant into various types of cells, including but not limited to live tumor cells, apoptotic tumor cells, dying tumor cells, immune cells, antigen-presenting cells.
Incorporation of the CpG ODN adjuvant into the lipid bilayer of cellular membranes and/or artificial membranes, including but not limited to plasma membranes, endosomes, lysosomes, endoplasmic reticular membranes, nuclear membranes, liposomes, micelles, lipoplexes, other types of lipid vesicles.
Chemical modification of CpG ODN for amphipathic function may include but are not limited to the following adjuncts linked to the 5′ and/or 3′ end of the CpG ODN: cholesterol or cholesteryl-modified molecules, neutral lipids, cationic lipids

### Primary Veterinary Cancer Cell Lines

The present invention further provides in part for establishing primary cancer cell lines derived from dissociated tumors. Establishing veterinary cancer cell lines comprise culturing cells in vitro and continuously growing in vitro-cultured cells established from surgically resected tumors from veterinary species. The cancer cell lines may be derived from but are not limited to the following veterinary species: canine, feline, equine, bovine.

The cancer cell lines may be derived from but are not limited to cutaneous tumors (i.e. melanoma, sarcomas and mast cell) and visceral tumors (i.e. colon, lung, prostate, kidney) or other tumors of the following general histologic types: carcinoma, sarcoma, myeloma, lymphoma, leukemia. The cancer cell lines may be characterized by proliferative potential, immunological and/or molecular tests to define species, tissue of origin and insure homogeneity of the final cell product.

### Allogeneic Veterinary Cancer Vaccine

The present invention provides in part for preparing an allogeneic vaccine. An allogeneic veterinary cancer vaccine comprises mixing defined primary veterinary cancer cell lines with an adjuvant for administration to a veterinary cancer patient. The veterinary cancer cell line in the vaccine comprises a single defined histologic type derived from the same species as the veterinary cancer patient. The veterinary cancer cell line in the vaccine may comprise a single cell line derived from a veterinary tumor or may be comprised of several separate cell lines of the same histologic type, derived from 2-5 individual surgically resected veterinary tumors.

The veterinary cancer cell line/lines in the vaccine are activated by treatment or exposure to ultraviolet irradiation in order to kill the tumor cells and to “activate” the tumor cells for processing by immune cells.

Allogeneic cancer vaccines may be combined with any or all of the following: veterinary species-optimized vaccine adjuvant, soluble stimulating immunologic reagents (see herein), and soluble inhibitory immunologic reagents (see herein)

### Hybrid Veterinary Cancer Vaccine

The present invention provides in part for a hybrid vaccine comprising autologous cells derived from a tumor within a subject mixed with defined cancer cell lines. The hybrid veterinary cancer vaccine comprises utilizing about 2 to 5 defined allogeneic veterinary cancer cell lines mixed with autologous dissociated tumor cells as described herein and an adjuvant for administration to a veterinary cancer patient. A dose of 10 million autologous tumor cells mixed with between 1-10 million cells from each allogeneic tumor cell line may to be utilized as a single vaccine dose to be injected into a to a veterinary cancer patient. This dose formulation may need to be adjusted to optimize immunization as studies progress.

Each veterinary cancer cell line in the vaccine may be of a single defined histologic type derived from the same species as the veterinary cancer patient. These histologic types include but are not limited to cutaneous tumors (i.e. melanoma, sarcomas and mast cell) and visceral tumors (i.e. colon, lung, prostate, kidney) or other tumors of the following general histologic types: carcinoma, sarcoma, myeloma, lymphoma, leukemia. The veterinary cancer cell line in the vaccine may comprise a single cell line derived from a veterinary tumor or may be comprised of several separate cell lines of the same histologic type, derived from 2-5 individual surgically resected veterinary tumors.

The veterinary cancer cell line/lines and the autologous tumor tissue in the vaccine may be treated by ultraviolet irradiation to kill the tumor cells making them more immunogenic for processing by immune cells.

### Veterinary Combination Immunotherapy

Effective cancer immunotherapy depends on a functional immune system in the cancer patient. Evidence in humans and animal models shows that tumor cells may express surface molecules that dampen the immune responses to cancer vaccines. One of these molecules, PD-L1, binds to a molecule on immune cells, PD-1 and inhibits functional activity by immune cells. Another molecule is CTLA-4 expressed on immune cells and which binds to CD80 or CD86 on antigen-presenting cells to dampen the immune response. Studies in humans and animal models demonstrate that blocking of the interaction between PD-L1 on tumor cells and PD-1 on immune cells or blocking the interaction between CTLA-4 with CD80 or CD86 can promote more effective anti-tumor immune responses during immunotherapy. Other studies in humans and animal models demonstrate that the addition of some soluble immune proteins, “cytokines” also can promote more effective anti-tumor immune responses.

Monoclonal antibodies specific for PD-L1, PD-1 and/or CTLA-4 in several veterinary species, including but not limited to canine and feline may be generated and administered in combination with other cancer treatments, such as with the vaccines described herein. Anti-PD-L1 and anti-PD-1 antibodies may be evaluated based on the ability to inhibit the interaction between PD-L1 and PD-1 in several veterinary species, such as by binding to or masking sites of interaction between the two. Anti-CTLA-4 antibodies may similarly be evaluated for the ability to inhibit the interaction between CTLA-4 and CD80 or CD86 in several veterinary species.

Monoclonal antibodies against PD-L1, PD-1 and/or CTLA-4 may be used as adjunctive therapy in combination with autologous, allogeneic or hybrid veterinary vaccines and veterinary species-optimized vaccine adjuvants. Treatment may be combined with traditional therapeutic approaches including cancer chemotherapy and/or surgical tumor resection.

Anti-PD-1 monoclonal antibodies, which are inhibitory for activated immune cells, may be used for therapy of autoimmune diseases in veterinary species, including but not limited to canine and feline.

### Kits

The present invention further provides kits for allowing or assisting in performing the methods described herein. The kits may comprise media and culturing materials to allow for establishing cell cultures. The kits may include enzymatic solutions to digest resected tumors, as well as surgical tools to resect a tumor. The kits may include vials for cryopreserving isolated cells. The kits may include adjuvants as described herein. Kits may also include defined cell lines to mix with isolated tumor cells as described herein.

### Examples

A mammal, such as a feline or a canine, with a cancerous growth is identified, the animal then sedated as necessary and the tumor is surgically removed by an attending veterinarian. Tumor cells are dissociated from other tissue by physical separation, as well as through partial digestion in a solution of collagenase, hyaluronidase and DNAse. Isolated tumor cells can then be further cultured, activated or mixed with other tumor derived cells, such as cells isolated from a secondary tumor within the animal or with defined cancer cells lines from the same species. Tumor cells can then be activated by exposure to ultraviolet radiation.

Activated tumor cells can then be mixed with an adjuvant comprised of single stranded unmethylated deoxynucleotides with phosphorothioate linkages. The adjuvant may comprise between one and 5 CpG motifs and may optionally comprise a stem-loop feature comprised of at least two AACGTT sequences. Adjuvant is then mixed with activated tumor cells to form a vaccine and administered to an animal suffering from a cancer. The administered activated tumor cells may be autologous, allogeneic or hybrids thereof to the animal receiving the vaccine. The animal receiving the vaccine may additionally be administered monoclonal antibodies or fragments thereof that bind PD-L1, PD-1 and/or CTLA-4 in the animal.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled. All documents referenced herein including patents, patent applications and journal articles and hereby incorporated by reference in their entirety.

What is claimed:

1. A method of processing a veterinary tumor vaccine, comprising:
resecting a tumor having tumor cells from a subject;
dissociating said tumor cells;
activating said tumor cells;
mixing said tumor cells with an adjuvant to formulate a tumor vaccine; and
injecting said tumor vaccine into an animal.

2. The method of claim 1, further comprising placing said tumor cells in a sterile container containing a sterile medium and a panel of antibiotics and anti-mycotics.

3. The method of claim 1, wherein the dissociating of the tumor cells further includes dissecting the tumor to remove necrotic tissue and residual normal tissue, placing the tumor in a digestion medium, and stirring the mixture until a single cell suspension is obtained.

4. The method of claim 3, wherein said digestion medium includes collagenase, hyaluronidase and DNAase.

5. The method of claim 1, wherein activating said tumor cells comprises treating the tumor cells with ultraviolet radiation.

6. The method of claim 1, further comprising cryopreserving a portion of said tumor vaccine prior to injecting said tumor vaccine.

7. The method of claim 1, further including shipping said tumor cell vaccine to a veterinarian for injection.

8. The method of claim 1, wherein the animal is the subject.

9. The method of claim 1, wherein the adjuvants comprises about 20 to 50 single stranded unmethylated deoxynucleotides.

10. The method of claim 9, wherein the adjuvant further comprises 1-5 CpG motifs and 1 to 4 AACGTT motifs.

11. The method of claim 9, wherein the adjuvant comprises one palindromic stem-loop motif comprising two AACGTT motifs.

12. The method of claim 1, wherein the dissociated tumor cells are cultured in vitro after UV radiation to activate tumor cells.

13. The method of claim 1, wherein the dissociated tumor cells are mixed with a defined cell line prior to being activated.

14. The method of claim 1, further comprising administering monoclonal antibodies against PD-L1, PD-1 and/or CTLA-4 to the animal.

15. A kit for processing veterinary tumor vaccines according to the method of claim 1.

16. A tumor vaccine comprising ultraviolet irradiated tumor cells and an adjuvant, wherein the adjuvant comprises about 20-50 single stranded phosphorothioate linked unmethylated deoxynucleotides.

17. The tumor vaccine of claim 16, wherein the adjuvant further forms a stem-loop structure comprised of at least two AACGTT motifs.

18. The tumor vaccine of claim 16, wherein the adjuvant further comprises between one and 5 CpG motifs.


SEQUENCE LISTING (1 sequence)

SEQ ID NO: 1 - DNA, 30 bp, Escheria Coli
tcgtcgtttaacgttgcgaggaacgttggg